(19) United States
(12) Patent Application Publication
(10) Pub. No.: US 2004/0067546 A1
(43) Pub. Date: Apr. 8, 2004

(54) Apparatus and method for purification and assay of neurites

(75) Inventors: Jay Leng, San Diego, CA (US); Russell Curry, San Diego, CA (US)

Correspondence Address: Raymond Y. Chan, Suite 128, 108 N. Ynez Ave., Monterey Park, CA, 91754, US

(73) Assignee: Innovative Cell Systems, LLC

(21) Appl. No.: 10/389,619

(22) Filed: Mar. 14, 2003

Related U.S. Application Data
(60) Provisional application No. 60/416,090, filed on Oct. 5, 2002.

(51) Int. Cl. C12M 1/12; C12N 5/00

(52) U.S. Cl. 435/30; 435/6; 435/297.5; 435/401; 435/402; 435/7.21

(57) ABSTRACT

An apparatus and method for purification and assay of neurites is useful for separation and analyses of extension organelles and/or protrusion of cells for purification, production, observation, and quantification of neurites in the neurobiology field. The present invention provides a pore-sized controlled porous filter membrane which outspace side surface is coated with a cell adhesion layer to form an adhesion surface, and combines the neuronal cells with the porous filter membrane in an aqueous environment under conditions in which outgrown neurites from cell bodies of the neuronal cells are attracted to and grow on the adhesion surface, wherein the outgrown neurites of the neuronal cells pass through pores provided in the porous filter membrane to the adhesion surface while each of the pores has a size smaller than the cell bodies of the neuronal cells so as to prevent the cell bodies of the neuronal cells passing through the pores and remaining on an opposing side surface of the porous filter membrane.

### CROSS REFERENCE OF RELATED APPLICATION

This is a regular application of a provisional application, application No. 60/416,090, filed on Oct. 5, 2002.

### BACKGROUND OF THE PRESENT INVENTION

1. Field of Invention

The present invention relates to an apparatus and method in the field of cell biology that is useful for separation and analyses of extension organelles and/or protrusions of cells, and more particularly to the purification, production, observation, and quantification of neurites in the neurobiology field.

2. Description of Related Arts

Neurites are cellular organelles generated by stimulated neuronal cell bodies. Neurites lengthen in response to specific contact stimuli and can ultimately mature into fully functional axons and dendrites. Compounds and biomolecules have been implicated in the ability to support the sprouting of neurites from a neuronal cell, a process also referred to as “neurite outgrowth”, which is essential in neural development and regeneration.

The characterization of neurite formation, growth, maturation and collapse or re-absorption is an area of intense interest, since these cellular processes are essential to interconnection of nerve cell bodies. Neurites are particularly interesting in relation to neuronal disorders, nerve tissue regeneration, and pharmacological research and therapeutic drug discovery and screening. As such, understanding the structure, function and expression of a molecule or molecules that mediate, separately or in concert with the complex molecular and cellular events in regulating neurite outgrowth in the nervous system is of paramount importance for both diagnostic and therapeutic uses.

The study of neurites and neurite growth, however, is severely hampered by the difficulty of isolating and purifying these minute organelles of the neuronal cells. More specifically, it would be highly desirable to be able to directly test neurite responses to many agents and combinations of agents. Yet the lack of means to isolate and purify sufficient neurite material, and the lack of uniform and highly reproducible methods for neurite characterization, have severely impeded an understanding of the role of these neurite organelles in development, injury and disease states.

One prior art assay for neutrophil chemotaxis which has been widely used for studying cell movement is the Boyden chamber assay. The Boyden chamber is composed of two parts which constitute an upper and a blind lower well. Chemoattractants are added to the lower well before placing the membrane which separates the lower from the upper well. The cells or pseudopodial protrusions that respond to the chemoattractants can be separated from the non-responding cells for further study.

The principle of the Boyden chamber assay of chemotaxis is further applied to separating cellular components such as pseudopodial protrusions from cell body or nucleus. One means of separating cellular components or protruding cellular organelles from the cell body, where they are of significant different size, is by providing a sieve or membrane with controlled pore size to effect the separation that permits the passage therethrough of the smaller sized cellular organelles but not the larger cell body. In the case of neural cells and their associated neurites, the vastly different size and shape suggests that some form of “oriented” filtration would suffice as a means of separation. This is because the rounded neural cell bodies generally are in the range of 20 microns in diameter, whereas neurites may extend many microns, but generally have cross sections of less than 0.8 microns. Thus, if neurites can be introduced or induced to traverse membrane pores substantially smaller than a neural cell body (for example, but not by limitation, 0.1 microns to 8.0 microns) but larger than the cross section of a neurite, then, neurites will appear in purified form on the side of the membrane distal to the surface on which neural cell bodies are deposited (hereafter the bottom surface of the membrane).

However, the means to accomplish the correct separation of the neurites from neuronal cell body is by no means obvious.

The use of a filter membrane in between chambers for cell culture and chemotaxis assay is known in the prior art. U.S. Pat. No. 4,912,057 discloses an apparatus comprised of a cell chamber containing a controlled-sized filter membrane sealed in between two cell culture plates for separating pseudopodia responding to chemoattractants in a chemotaxis assay. The pseudopodia, which are active and adventitious extensions of living cells, can be isolated by means of membrane separation. In this process, cells are placed in a upper chamber and the chemoattractant solution is situated in the lower chamber with a porous membrane sealed or fixed in between the two plates with a locking device, as shown in FIG. 1. Pseudopodia can pass through sufficiently large membrane pores in response to chemoattractant solutions placed below the membrane but in contact with membrane bottom. The directionality of the pseudopods, by which they traverse the membrane pores, is a function of the chemoattractant agent. The cells in the upper chamber may be swabbed from the membrane and rinsed out of the chamber, leaving the detached pseudopodia still attached to the membrane. The emergent pseudopods can be studied directly on the membranes; for instance, the pseudopods may be quantified by first staining them, washing the membrane free of excess stain and then destaining the pseudopods into a recipient liquid for colorometric assay. The pseudopodia, fully separated from the cell bodies on the other side of the membrane, may be used by a detergent solution and the solubilized fraction assayed for its contents. Absent this isolation or purification, assessing the properties and responses of the pseudopodia would be either more difficult or not possible. Devices for this purpose are prior arts, and are comprised of a one or an array of chamber(s), wherein the chamber is open at one end. Cell suspensions are introduced into the chamber at the open end. In each case and as described above, the cells dispensed into the open end of the chambers settle onto the membrane secured across the bottom of the chamber. The membrane bottom surface is then contacted with solutions containing chemoattractant solution. Because the solution containing the cells does not contain chemoattractant, whereas the solution contacting the underside of the membrane contains chemoattractant, a pseudopod chemoattractive gradient is established through the membrane pores and provides directional growth of the pseudopods.

The membrane-based pseudopod purification suggests that neurites may also be purified through using membrane with controlled pore sizes. However, neurites are threadlike cellular extensions capped by a complex sensing apparatus called the “growth cone”. Unlike pseudopodia, which can provide their own attachment signals and secrete their own anchoring molecules, the extension of neurites requires sensing of appropriate macromolecule signals by the growth cones. Neurites are also very delicate, unlike pseudopodia, so for both signaling and recovery purposes they must attach securely to the underside of the membrane. Unlike pseudopods, neurites require contact with specific signal molecules in order to extend, and will not attach at all to the unmodified synthetic membranes which support pseudopods.

Also well known in the art is that the adhesion of cells to a surface is a multi-step process, consisting of initial attachment (characterized by weak binding and little cell shape change) followed by cell spreading (which produces stronger binding of cells to the substrate) (Grinnell, F., “Cellular Adhesiveness and Extracellular Substrata”, Internat. Rev. Cytology 53:65-144 (1978)). The initial attachment can be mediated by non-specific mechanisms such as charged surfaces (Grinnell, F., “Cellular Adhesiveness and Extracellular Substrata”, Internat. Rev. Cytology 53:65-144 (1978) and Microcarrier Cell Culture. Principles and Methods, Pharmacia Fine Chemicals, Uppsala, Sweden, pages 5-33 (1981)). In contrast to initial attachment, cell spreading seems to require the presence of specific receptor-ligand interactions between cell surface receptors and certain cell adhesion glycoproteins, such as fibronectin, laminin, and collagens (Kleinman, H. K., Luckenbill-Edds, F. W. Cannon, and G. C. Sephel, “Use of Extracellular Matrix Components for Cell Culture”, Anal. Biochem. 166:1-13 (1987)). All three types of these glycoproteins have been purified and added to tissue culture surfaces to promote cell adhesion and cell growth (Kleinman, H. K., Luckenbill-Edds, F. W. Cannon, and G. C. Sephel, “Use of Extracellular Matrix Components for Cell Culture”, Anal. Biochem. 166:1-13 (1987)). Studies have shown that a coating of gelatin or denatured collagen on microcarriers facilitates the attachment and growth of mammalian cells (Microcarrier Cell Culture. Principles and Methods, Pharmacia Fine Chemicals, Uppsala, Sweden, pages 5-33 (1981)).

It is well documented that the neurites attach strongly on surfaces coated with extracelluar matrix (ECM) proteins such as fibronectin, laminin and/or collagens. Thus, the outgrowth of the neurites can be directed surfaces coated with ECM proteins. The prior art, U.S. Pat. No. 4,912,057, discloses a controlled-pore sized porous filter constructed of polycarbonate coated with Type I or Type II collagen, fibronectin or laminin. However, the filter's coating is non-directional and used for cell attachments on both sides of the filter. U.S. Pat. No. 5,512,474 discloses a non-porous cell culture support coated with positively charged molecules and a cell adhesion factor selected from the group consisting of fibronectin, laminin, collagen, vitronectin and tenacin, and active fragments and synthetic analogs having a cell binding domain thereof on the surface for cell attachment.

### SUMMARY OF THE PRESENT INVENTION

It is a main objective of the present invention to provide an apparatus and method for purification and assay of neurites to rapidly and conveniently contact neural cells and neurites with diverse panels of organic and inorganic substances, substantially enhancing the possibility of new drug discovery. The present invention further provides neurites useful in all aspects of their physical, physiological, biological and biochemical characterization.

It is another objective of the present invention to provide an apparatus and method for purification and assay of neurites, which is useful for separation and analyses of extension organelles and/or protrusion of cells for purification, production, observation, and quantification of neurites in the neurobiology field.

In order to accomplish the above objectives, the present invention provides a cell culture apparatus specifically adapted for the propagation, purification and analysis of neurites. The neurite culture apparatus comprises a culture cylinder or multi-well plate sealed underneath with a pore-size controlled porous filter membrane, which one-side provides an outspace side surface coated with cell adhesion factors.

In one embodiment, the control-pore sized filter membrane is treated with extracellular matrix (ECM) proteins, which coat the underside or bottom of the membrane and into the pores of the membrane, providing either a “step gradient” or, depending on the method of wetting the membrane, a more gradual gradient of ECM agents, an example of which is laminin, which the cell may track from the time it initially settles at or near a pore. Neurites emerging from the neuronal cell body, thus, traverse the membrane pores, emerging and subsequently attaching and tracking across the bottom surface of the membrane. The membranes are affixed to the bottom edge of chambers intended to hold aliquots of neuronal test cells. Depending on the application, the apparatus can be a single chamber, a set of chambers, or a multi-well plate comprising from a few to hundreds of individual chambers.

The chambers are arranged to fit into a receiver vessel, which in use contains a solution adapted to contact the bottoms of the membranes. The solutions may contain nerve growth factors essential to the propagation of neuronal cells, and in addition other nutrients and agents for the support of the neurites as they emerge from the pores openings on the underside of the membrane.

It is understood that while a preferred embodiment of the present invention uses either purified preparations of natural ECM, other synthetic or natural components of ECM, or synthetic or natural biological compounds, inorganic or organic polymers or small molecules and pharmacological agents of many types might be introduced into the membrane coating/gradient, or be provided in the solution contacting the lower membrane surface, or be placed in the upper cell-containing chamber, any of which might affect the performance of the present invention.

The present invention further provides a method of attracting and growing anchorage-dependent neurites on a coated surface of a cell culture system, comprising the steps of:

(a) providing a pore-sized controlled porous filter membrane coated, on the bottom side of the culture apparatus, cell adhesion factor, and

(b) combining the neuronal cells with the coated membrane in an aqueous environment under conditions in which the neurites of the neuronal cells are attracted to and grow on the coated underside surface.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a sectional view illustrating a cylinder or chamber which encloses a cell culture chamber closed at its bottom end by a porous membrane according to a preferred embodiment of the present invention.

FIG. 2 is a schematic view illustrating a living neural cell body deposits on the porous membrane according to the above preferred embodiment of the present invention.

FIG. 3 is a partial perspective view illustrating chambers of a ninety-six well plate with each individual well sealed with ECM coated porous membrane such that the porous membrane contacts neurite sustaining fluid media (containing nerve growth factor) in the reservoir according to above preferred embodiment of the present invention.

FIG. 4 illustrates quantification of neurite formation of NIE-115 neuroblastoma on Laminin according to the above preferred embodiment of the present invention.

### DETAILED DESCRIPTION OF THE PREFERRED EMBODIMENT

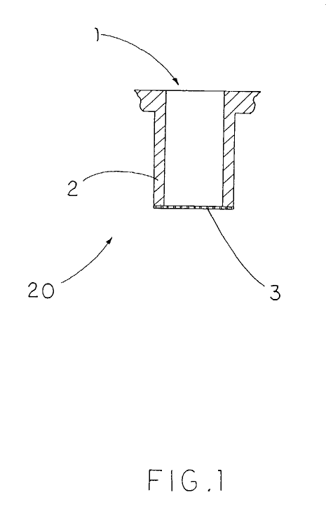

Referring to FIGS. 1 to 4 of the present invention, an apparatus and method for purification and assay of neurites according to a preferred embodiment of the present invention is illustrated. The cell culture apparatus, which is specifically adapted for propagation, purification and analysis of neurites, comprises a neurite culture device which is a single unit of neurite culture insert 20 comprising a culture cylinder or chamber 2 defining a cell culture chamber 1 enclosed therein and a pore-size controlled porous filter membrane 3 which has pores 31 distributed thereon, is sealed underneath the culture cylinder 2. The porous filter membrane 3 is one-side coated on the outspace side surface, the underside or the bottom with cell adhesion factors.

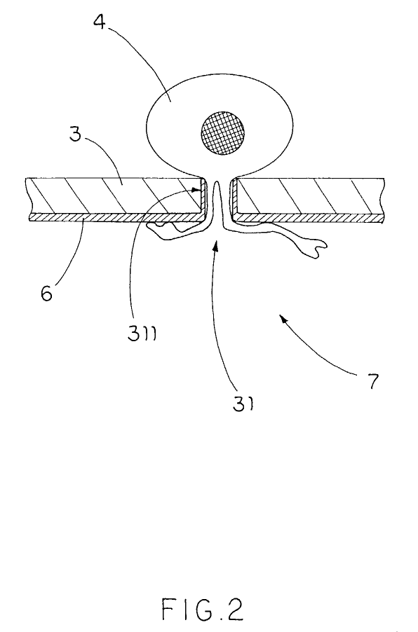

As shown in FIG. 2, a living neural cell body 4 is deposited on the porous filter membrane 3, in which a cell senses extracellular matrix (ECM) layer 6 is coated on an underside of the porous filter membrane 3 and a pore internal surface 311 of the respective pore 31, resulting in the budding of neurites 7 that traverse the pore 31 and into the ECM coated bottom surface of the porous filter membrane 3.

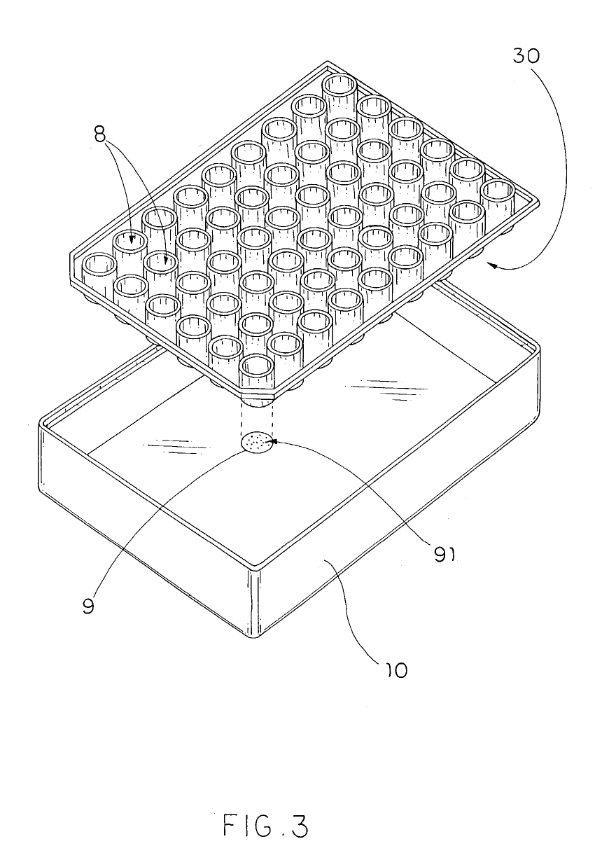

In an alternative mode of the preferred embodiment, the neurite culture device comprises a multi-well plate 80 having the bottom of each well sealed with a pore-size porous filter membrane 9 with the bottom surface coated with the cell adhesion factor to provide a surface for the outgrown neurites to traverse and attach onto the underside that separated from the side of a neuronal cell body 4 is shown in FIG. 3. In which, each chamber 8 of the (ninety-six) multi-well plate 80 with its individual well sealed with ECM coated with the porous filter membrane 9, such that the porous filter membrane 9 contacts neurite sustaining fluid media (containing nerve growth factor) in a reservoir 10.

In other words, a number of neurite culture devices, which will be alternatively referred herein to as neurite culture insert 20 and/or neurite culture plate 80, exist for culturing the anchorage-dependent neurites, and the present invention is not dependent upon any particular type or configuration of neurite culture devices. The “neurite surface”, the “underside surface” or the “bottom surface” of the present invention, i.e., the outspace side surface of the neurite culture cylinder 2 or neurite culture plate 80 that is adapted to physically attract, contact and support outgrowing neurites, bears an effective and stable cell adhesion factor or in combination of a number of cell adhesion factors.

In view of the above preferred embodiment, the present invention provides means to rapidly and conveniently contact neuronal cells and neurites with diverse panels of organic and inorganic substances and/or macromolecules, substantially enhancing the possibility of new drug discovery. Practically, neurite culture devices can be provided in any suitable form, for instance, as membranes, tubes, microtiter wells, columns, hollow fibers, roller bottles, plates, dishes, and solid, hollow, or porous beads. The filter membrane of the neurite culture device is coated at one side the cell adhesion factor for the outgrown neurite to be attracted and attached. Accordingly, the present invention is capable of providing neurites useful in all aspects of their physical, physiological, biological and biochemical characterization.

The pore size controlled porous membrane 3, 9 of the neurite culture devices of the present invention can be prepared having a pore size ranging from 0.1 to 10 microns, preferably ranging from 1 to 8 microns and most preferably at about 2 to 5 microns providing a pass through for the outgrown neurite, typically with a cross-sections of less than 0.8 microns, to traverse through the pores while retaining the neuronal cell body 4, generally of 20 microns in diameter, in the upper chamber or on the upper surface of the neurite culture chamber 1, 8.

A wide variety of materials can be employed as the porous filter membrane 3, 9, the primary considerations being that they are preferably neither soluble or swellable in water. Suitable porous membrane materials provide a surface that exhibits an optimal combination of such properties as rigidity, surface area, ease of preparation and use, and cost.

Preferred membrane materials are rigid, i.e., do not swell or expand appreciably, and thus, maintain the pore size in an aqueous environment. Preferred membrane materials, for instance, expand less than about fifty percent, and preferably less than about twenty percent, in any dimension when placed from the dry state into isotonic saline.

Preferred membrane materials are synthetic polymers, including oligomers, homopolymers, and copolymers resulting from either addition or condensation type polymerizations. Examples of suitable addition type polymers include, but are not limited to, acrylics such as those polymerized from methyl acrylate, methyl methacrylate, acrylic acid, methacrylic acid, acrylamide, and methacrylamide; vinyls such as styrene, vinyl chloride, vinyl pyrrolidone, and vinyl acetate; polymers formed of ethylene, propylene, and tetrafluoroethylene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, and poly(ethylene terephthalate). Other suitable materials include ceramics, e.g., silicon nitride, silicon carbide, zirconia, and alumina, as well as glass, silica, and the like.

The control-pore sized porous filter membrane 3, 9 is treated or coated with the extracellular matrix (ECM) proteins, which coat the underside or bottom of the porous filter membrane 3, 9 and into the pores 31, 91 of the porous filter membrane 3, 9, providing either a “step gradient” or, depending on the method of wetting the membrane, a more gradual gradient of ECM agents, an example of which is laminin, which the cell 4 may track from the time it initially settles at or near a pore.

Neurites emerging from the neuronal cell body 4, thus, traverse the membrane pores 31, 91, emerging and subsequently attaching and tracking across the bottom surface of the porous filter membrane 3, 9, as shown in FIG. 2, wherein the porous filter membranes 3, 9 are affixed to the bottom edge of chambers 1, 8 adapted to hold aliquots of neuronal test cells 4. Depending on the application, the apparatus can be a single chamber 1 of a neurite culture insert 20, a set of chambers, or a multi-well plate 80 comprising from a few to hundreds of individual chambers 8.

The chambers 1, 8 are designed to fit into the reservoir 10 such as a receiver container which in use contains a solution intended to contact the bottoms of the porous filter membranes 3, 9, as shown in FIG. 3. According to the preferred embodiment, such solutions provide an aqueous environment and may contain nerve growth factors essential to the propagation of neuronal cells 4, and in addition other nutrients and agents for the support of the neurites as they emerge from the pores openings on the underside of the porous filter membrane 3, 9.

It is understood that while the preferred embodiment of the present invention uses either purified preparations of natural ECM, other synthetic or natural components of ECM, or synthetic or natural biological compounds, inorganic or organic polymers or small molecules and pharmacological agents of many types might be introduced into the membrane coating/gradient, or be provided in the solution contacting the lower membrane surface, or be placed in the upper cell-containing chamber, any of which might affect the performance of this invention.

As described in more detail below, the cell adhesion factors for attracting neurite growth cones and stimulating and attaching the bodies of outgrowing neurites is preferably one or more proteins of the extracellular matrix (ECM) protein, or a preparation of ECM. The neurite culture device comprising the present invention improves the attachment and growth of the “anchorage dependent” neurites through favorable modification of the membrane surface, i.e. neurites are known to attach to substrates in a manner to some degree similar to “anchorage-dependent” cells. Such cells generally need to attach to a support surface and spread in order to grow and divide (Grinnel, F., “Cellular Adhesiveness and Extracellular Substrata”, Int. Rev. Cytology 53:65-114 (1978)). Like cells, neurite attachment, growth and guidance on surfaces is mediated by polypeptides thereof. However, attachment of cells and extension and attachment of neurites to porous and non-porous surfaces and membranes is known in the art to also occur by means of static or induced charges. By example, but not by limitation, these could be poled polytetrafluoroethylene (PFE) (Vallenti, R. F. et al, Biomaterials 1989;13, 183-190), poled polyvinylidene fluoride (PVDF) (Valenti, R. F. et al, Brain Research 1989:480, 300-304) or fluoroethylenepolypropylene (FEP) (Makohliso, S. et al, J. Biomed Materials Res., 1993;27(8), 1075-1085. The electrostatic charges may be an intrinsic property of the membrane composition (as recited herein above), or may be induced asymmetrically to one side of the membrane, for instance by plasma or corona discharge (Makohliso, ibid). It is also clear to one skilled in the art that such implantation could be modified by masking of a part of the membrane surface, or otherwise directing patterns of deposition of positively charged molecules conducive to neurite outgrowth and attachment. The charge properties conducive to neurite extension and attachment may be further adjusted and modulated by additional components, such as dopants ((Preznayna, L., et al, 1991;24,5283-5287) or applied coatings of positively charged molecules.

Thus one alternative mode to use of ECM and ECM-related peptides to provide neurite attachment and outgrowth in the present invention is the use of membranes variously treated by positively charged molecules or static charges or combinations. The present invention, in addition to properties intrinsic to any of the several types of membranes recited herein, would include any and all inventive embodiments wherein was used neurite attachment agents, including membrane electrets, induced membrane electrets and electric fields, membrane dopants, membranes coated with positively charged molecules, co-coatings of positively charged molecules, electrets and/or component proteins of the ECM, in any and all isotropic and anisotropic useful combinations of the foregoing agents.

Alternatively, positively-charged molecules employed on the bottom surfaces of the present invention can promote cell attachment to the surface by promoting ionic binding between the positively-charged molecules and cell constituents, such as negatively-charged glycoproteins and phospholipids carried on cellular surfaces.

In another alternative mode, the neurite surface can be coated by combining the use of cell adhesion factors and positively-charged moieties. The present invention provides the opportunity for either or both types of neurite attachment, likely by a mechanism involving both receptor-mediated and non-receptor-mediated (i.e., charger-elated) cell attachment.

“Cell adhesion factor”, as used herein, refers to a molecule that mediates the adherence of cells, via the cell's receptors, to a support surface, e.g. in order to increase the rate at which such cells grow and spread on that surface. Preferably, suitable cell adhesion factors include cell adhesion proteins, cell adhesion protein peptide fragments, and synthetic peptide analogs. Examples of preferred cell adhesion factors useful with the present invention include such cell adhesion proteins as laminin, fibronectin, collagens (all types), vitronectin, and tenascin; cell adhesion peptides such as the cell attachment domain of fibronectin identified as the tripeptide (RGD) and the cell attachment domain of laminin identified as the pentapeptide (YIGSR) of laminin; as well as other binding domains of these and other cell adhesion proteins and functional synthetic analogs thereof.

Cell adhesion proteins typically have one or more domains that mediate binding to cell surface receptors. According to the preferred embodiment, these cell attachment domains include specific amino acid sequences that can be chemically synthesized to produce cell adhesion peptides that possess the cell attachment properties of the intact cell adhesion proteins. Two examples of such cell adhesion peptides are the tripeptide (RGD or arg-gly-asp) sequence present in fibronectin and the pentapeptide (YIGSR or tyr-ile-gly-ser-arg) sequence present in laminin: Ruoslahti, E. and M. Pierschbacher, “Arg-Gly-Asp: A versatile Cell Recognition Signal,” Cell 44:517-518 (1986); Pierschbacher, M. D. and E. Ruoslahti, “Cell Attachment Activity of Fibronectin can be Duplicated by Small Synthetic Fragments of the Molecule,” Nature 309:30-33 (1984). Graf, J. et al., “Identification of an Amino Acid Sequence in Laminin Mediating Cell Attachment, Chemotaxis, and Receptor Binding,” Cell 48:989-996 (1987).

Cell adhesion proteins are primarily those that are naturally occurring and quite large, with molecular weights above about 100,000 daltons. Cell adhesion peptides generally are short amino acid sequences derived from or functionally analogous to the binding domains of the cell adhesion proteins.

Cell adhesion factor is used at a surface density sufficient to promote initial cell attachment and to stabilize attachment of the cells to the surface. The density of the cell adhesion factor will vary and will depend in part upon such factors as the configuration of the neurite culture device, the material with which the porous membrane is made.

According to the preferred embodiment, a sufficient density of cell adhesion factor should be coated onto the underside or the bottom of the neurite surface to promote neurite attachment and growth. For example, the density of cell adhesion factor will desirably range from about 0.01 to about 1000 picomoles of factor per square centimeter of the bottom surface.

In the preferred embodiment with cell adhesion proteins, the desirable range is from about 0.01 picomoles to about 100 picomoles, and with cell adhesion peptides, the desirable range is from about 0.1 picomole to about 1000 picomoles per square centimeter of bottom surface.

Alternatively, the coating, which can be a cell adhesion factor for the neurites, can include, or itself be provided by, one or more other materials that may not be considered either positively charged molecules or a cell adhesion factor as defined herein. As consideration for different types of cell adhesion factors, such as gelatin and one or more cell attachment peptides, can be coated onto the same neurite or bottom surface.

Cell adhesion factors promote cell attachment by binding to specific receptors on the cell surface, and some cell types have receptors for more than one type of cell adhesion factor; Buck, C. A. and A. F. Horwitz, “Cell Surface Receptors for Extracellular Matrix Molecules,” Ann. Rev. Cell Biol. 3:179-205 (1987). Immobilizing different types of cell adhesion factors upon the same bottom neurite surface may allow the binding of more receptors on each neurite surface than would occur with a simple type of coated cell adhesion factor, thereby possibly resulting in faster and stronger neurite attachment to the bottom surface.

The present invention further provides a method of attracting and growing anchorage-dependent neurites on a coated surface of a cell culture device, comprising the steps of:

(a) providing a pore-sized controlled porous filter membrane 3, 9 which bottom side is coated with a cell adhesion factor 6 to form a coated adhesion surface of the porous filter membrane 3, 9; and

(b) combining the neuronal cells 4 with the porous filter membrane 3, 9 in an aqueous environment under conditions in which neurites of neuronal cells are attracted to and grow on the coated adhesion surface.

According to the preferred embodiment, the method further comprises the steps of:

(c) culturing the neuronal cells 4 in the upper chamber 1, 8 with nerve growth factor (NGF) and other nutrients in the culture media; and

(d) separating the outgrown neurites from the neuronal cell bodies 4 by scraping the upper chamber surface so that neurites are purifiable from the bottom side of the porous filter membrane 3, 9.

According to the preferred embodiment, the method may further comprise a step of:

(e) analyzing the neurites on the porous filter membrane 3, 9 with immunohistochemical staining assays.

Alternatively, the method may comprise the steps of:

(f) analyzing subcellular contents of the neurites by isolating the lysate of the purified neurites free of neuronal cell bodies.

The neurite contents may change under different physiological, electrophysiocal, pathological and pharmacological conditions. In analyzing the neurotropical drugs and therapeutical modalities, the neurite growth and/or neurite contents and other physiological parameters can be dissected and tracked in a timely manner and therapeutics pharmacokinetics followed in a time course.

In order to further illustrate the distinctive features of the present invention, several examples are described as follows, which are intended to illustrate, but not limit, the scope of the invention.

### EXAMPLE 1

### Neural Cell Culture

Healthy mouse NIE-115 neuroblastoma cells of low passage (<p30) were cultured in DMEM culture media with 10% FBS to 60-70% confluent. Three 100-mm dishes of the 60-70% confluent NIE-115 cells were split into nine 100-mm dishes containing 7 ml of the DMEM complete 10% FBS (no sodium pyruvate) and cultured for 3 to 4 days until cells are of 70-80% confluent and then serum-starved overnight in DMEM with 0.2% BSA without serum to induce differentiation and neurite extension. The procedure of neural cell culture shall yield enough cells for approximately 8-10 24 mm porous filter membranes and 150-200 μg of total purified neurite proteins.

### Preparation of Porous Membranes

A solution of 10 μg/ml purified laminin was prepared in sterile PBS containing no cations from Sigma stock at 1 mg/ml. The prepared ECM solution (2 ml) was added to bottom chamber. The Corning 6-well 24 mm plate with 3.0 μm pore-sized membrane was immersed in the ECM solution and coated for 2 hours at 37° C.

### EXAMPLE 2

### Preparation of Cells and Induction of Neurite Extension

Thirty minutes before completion of ECM coating of the neurite culture wells, the neuronal cells are removed from the culture dishes with dilute detachment buffer. The neuronal cells are resuspended at 3×10⁶ cells/ml in warm migration/adhesion buffer containing 0.2% RIA grade BSA.

Exactly 2.5 ml of warm migration/adhesion buffer is added to each well of a 6-well dish. The Corning Plate with coated porous filter membrane is removed from the Laminin solution and excess buffer is shaken off (the membrane need not be rinsed). Exactly 1.5 ml of the cell suspension is added to the upper chamber/membrane and immediately placed into a well of a 6-well dish containing 2.5 ml of migration/adhesion buffer. The neuronal cells are allowed to extend neurites to the lower chamber for 4-72 hours at 37° C. 16 hours is found to be the best and most convenient time point for staining. The porous filter membranes are removed and gently rinsed in excess PBS and then fixed in 100% ice cold methanol for 20 minutes, or alternatively, directly stained in crystal violet solution. Uptake of crystal violate is quantitated via OD measurement.

### EXAMPLE 3

### Preparation of Purified Neurites with 1% SDS Lysis Buffer

To isolate neurite proteins from the bottom of the porous filter membrane, the porous filter membrane is removed from the methanol fixitive, gently rinsed in excess PBS at room temperature and gently shaken to remove excess PBS. A cotton swab is used to remove cell bodies from the top of the porous filter membrane. To obtain pure neurites, it is important to remove all of the cell bodies and debris from the top, especially to remove them at around the edges of the porous filter membrane where it attaches to the plastic chamber. Gently rinse the filter in excess PBS to remove all cell debris and repeat the once. Dry the outer edges of the chamber with a kimwipe with extra care to avoid touching the bottom of the porous filter membrane containing the neurites. Place a drop of 100-200 μl of the 1% SDS Lysis Buffer (pH 7) containing the 1 mM vanadate and protease inhibitors on 3×3 piece of flat parafilm and place bottom of well onto the Lysis Buffer drop so that it covers entire bottom surface. Scrape neurites from the porous filter membrane with a cell scraper and place well back into drop of Lysis Buffer and scrape again as above. Remove remaining Buffer on the bottom and top of the porous filter membrane with a pipet and add back to the original drop of Lysis Buffer. The same drop of Lysis Buffer can be used to collect the neurite lysates from the bottoms of each coated neurite culture well to obtain highly concentrated neurite proteins into a small volume for further protein analysis. Upon finishing collection of the neurites in the Lysis Buffer, boil the lysates for 10 minutes.

### EXAMPLE 4

### Preparation of the Back (Top) of Polarized Cell

To isolate proteins from the top of the porous filter membrane (i.e. neuronal cell bodies), remove the porous filter membrane from fixitive and gently rinse in excess PBS at room temperature. Shake off excess PBS and use a cotton swab to remove neurites from the bottom of the porous filter membrane. As discussed previously, it is important to remove all debris from the bottom of the porous filter membrane prior to lysis. Rinse in excess PBS to remove all debris and repeat the step using a new cotton swab. Dry the inner and outer edges of the chamber with a kimwipe with extra care not to touch the top of the porous filter membrane containing the cell bodies. Cover the top of the porous filter membrane with 175 μl of Lysis Buffer and scrape the cell bodies from the porous filter membrane with a cell scraper. Remove Lysis Buffer from the top of the porous filter membrane with a pipet with the tip being cut off and place into a 1.5-ml eppendorf tube and boil for 10 minutes. Approximately 210 μg of proteins were collected from the lysate of neuronal cell bodies from each 24 mm well. The same Lysis Buffer can be used for each porous filter membrane to collect the cell lysates of the neuronal cell bodies.

### EXAMPLE 5

### Quantification of Neurite Formation of NIE-115 Neuroblastoma on Laminin

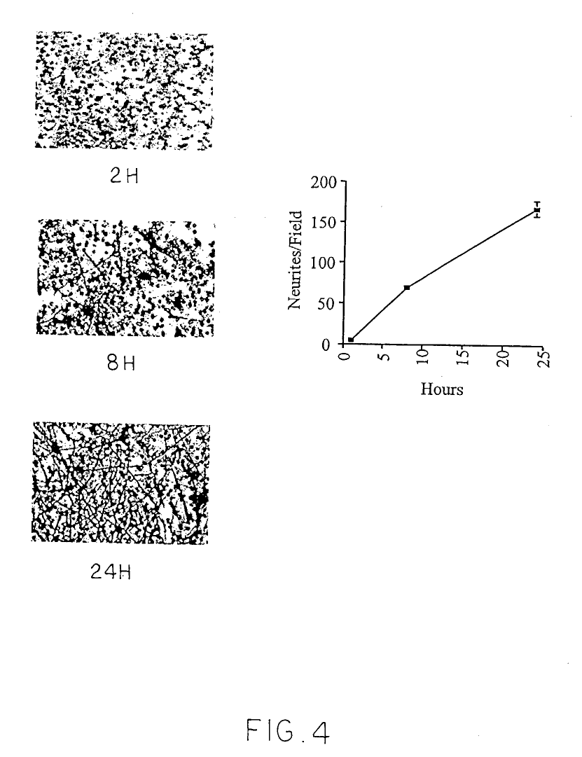

NIE-115 neuroblastoma cells were allowed to form neurite extension on a laminin coated transwell insert (3 μm pore size) for 2-24 hours. Approximately 300,000 living cells were added to the top of each chamber. After removing the cell bodies by swabbing, as described in the Example 3, the neurites on the bottom side of the membrane were stained with 0.1% Crystal violet solution (left panel). The number of neurites per porous filter membrane were counted with inverted microscope in three randomly selected fields (right panel). FIG. 4 shows a time course of the progression of neurites through the bottom of a porous membrane of 3.0-micron pore size as a function of time. Also shown is the abundance of the neurite material emerging as a function of time.

While a preferred embodiment of the present invention has been described in these Examples, it should be understood that various changes, adaptations and modifications may be made therein without departing from the spirit of the invention and the scope of the appended claims.

Having described the preferred embodiment and its alternatives of the present invention, it will appear to those ordinarily skilled in the art that various modifications may be made to the disclosed embodiment, and that such modifications are intended to be within the scope of the present invention. Although the invention has been described with reference to the above examples, it will be understood that modifications and variations are encompassed within the spirit and scope of the invention. Therefore, the present invention includes all modifications encompassed within the spirit and scope of the following claims.

What is claimed is:

1. A neurite culture apparatus for culturing neuronal cells, comprising;
a filter membrane having one or more pores each having a size adapted to allow protrusions of cellular organelles of each of the neuronal cell to pass through without allowing a cell body of the neuronal cell to pass therethrough; and
a cell adhesion layer provided on an outspace side surface of said filter membrane to form an adhesion surface being in contact with an aqueous environment for attaching said cellular organelles.

2. The apparatus, as recited in claim 1, wherein said pore of said filter membrane has a size of 0.8 to 10 microns.

3. The apparatus, as recited in claim 1, wherein said filter membrane further has another opposing side surface for the cell body of said neuronal cell positioning thereon.

4. The apparatus, as recited in claim 1, wherein said cell adhesion layer is a material on said outspace side surface of said filter membrane, wherein said material is selected from a group consisting a cell adhesion factor, a positively-charged molecule, an electrostatic charged material, an induced electrostatic charged material, a native electrostatic charged material and a combination of said cell adhesion factor, said positively-charged molecule, and said electrostatic charged material.

5. The apparatus, as recited in claim 4, wherein said cell adhesion factor is selected from a group consisting of fibronectin, laminin, collagen, vitronectin and tenascin, and active fragments and synthetic analogs having a cell binding domain thereof, and a combination thereof.

6. The apparatus, as recited in claim 4, wherein said cell adhesion factor is a laminin.

7. The apparatus, as recited in claim 4, wherein said positively-charged molecule is selected from a group consisting of polylysine, chitosan, poly(ethyleneimine), and acrylics polymerized from acrylamide or methacrylamide and incorporating positively-charged groups in the form of primary, secondary, or tertiary amines, or quaternary salts and a combination thereof.

8. The apparatus, as recited in claim 4, wherein said electrostatic charged material is selected from a group consisting of poled polytetrafluoethylene (PFE), poled polyvinylidene fluoride (PVDF), fluoroethylenepolypropylene (FEP) and a combination thereof.

9. The apparatus, as recited in claim 4, wherein said electrostatic charged material is created by electrostatic charges induced asymmetrically of the membrane composition by means of plasma or corona discharge.

10. The apparatus, as recited in claim 4, wherein said positively-charged molecule, said electrostatic charged material, and/or said cell adhesion factor are covalently bound to said outspace side surface of said filter membrane.

11. The apparatus, as recited in claim 4, wherein said positively-charged molecule, said electrostatic charged material, and/or said cell adhesion factor are covalently bound to one another while said positively-charged molecule and/or electrostatic charged material is covalently bound to said outspace side surface of said filter membrane.

12. The apparatus, as recited in claim 4, wherein said positively-charged molecule, said electrostatic charged material, and/or said cell adhesion factor are covalently bound to one another while said cell adhesion factor is covalently bound to said outspace side surface of said filter membrane.

13. The apparatus, as recited in claim 4, wherein said positively-charged molecule is provided in a form of a stable coating noncovalently bound on said outspace side surface of said filter membrane.

14. The apparatus, as recited in claim 4, wherein said cell adhesion factor is provided in a form of a stable coating noncovalently bound on said outspace side surface of said filter membrane.

15. The apparatus, as recited in claim 4, wherein said positively-charged molecule, said electrostatic charged material, and/or said cell adhesion factor are provided in the form of a stable coating noncovalently bound on said outspace side surface of said filter membrane.

16. The apparatus, as recited in claim 1, wherein said filter membrane is selected from a group consisting of polycarbonate, polystyrene and polypropylene.

17. The apparatus, as recited in claim 1, wherein said cell adhesion layer extends to a pore internal surface of each of said pores of said filter membrane.

18. A neuronal cell culturing apparatus, comprising:
a neurite culture device defining at least a cell culture chamber;
a pore-size controlled porous filter membrane, which is sealed at one end of said cell culture chamber, having one or more pores each having a size adapted to allow protrusions of cellular organelles of a neuronal cell to pass through without allowing a cell body of the neuronal cell to pass therethrough; and
a cell adhesion layer coated on an outspace side surface of said porous filter membrane to form an adhesion surface for rapidly and conveniently contacting neuronal cells and neurites.

19. The apparatus, as recited in claim 18, wherein said cell culture device comprises at least a culture cylinder defining said cell culture chamber therein, wherein said porous filter membrane is affixed to one end of said culture cylinder while said adhesion surface facing outside.

20. The apparatus, as recited in claim 18, wherein said cell culture device comprises a multi-well neurite culture plate providing a plurality of cell culture chambers, wherein one end of each of said cell culture chamber is sealed with said porous filter membrane to provide said adhesion surface facing outside.

21. The apparatus, as recited in claim 19, wherein said adhesion surface is in contact with an aqueous environment.

22. The apparatus, as recited in claim 20, wherein said adhesion surfaces are in contact with an aqueous environment.

23. The apparatus, as recited in claim 21, wherein said cylinder is disposed on a reservoir containing a neurite sustaining fluid media until said adhesion surface of said porous filter membrane contacts with said neurite sustaining fluid media.

24. The apparatus, as recited in claim 22, wherein said multi-well neurite culture plate is disposed on a reservoir containing a neurite sustaining fluid media until said adhesion surfaces of said porous filter membranes contact with said neurite sustaining fluid media.

25. The apparatus, as recited in claim 18, wherein said pore of said porous filter membrane has a size of 0.1 to 10 microns.

26. The apparatus, as recited in claim 18, wherein said cell adhesion layer is a material coated on said outspace side surface of said porous filter membrane, wherein said material is selected from a group consisting a cell adhesion factor, a positively-charged molecule, an electrostatic charged material, and a combination of said cell adhesion factor, said positively-charged molecule and said electrostatic charged material.

27. The apparatus, as recited in claim 18, wherein said cell adhesion layer is an extracellular matrix (ECM) layer.

28. The apparatus, as recited in claim 18, wherein said cell adhesion layer extends to a pore internal surface of each of said pores of said porous filter membrane.

29. The apparatus, as recited in claim 18, wherein said porous filter membrane is made of a rigid material which is neither soluble nor swellable in water.

30. The apparatus, as recited in claim 26, wherein said outspace side surface of said porous filter membrane is coated with an extracellular matrix (ECM) protein to form said adhesion surface.

31. The apparatus, as recited in claim 23, wherein said neurite sustaining fluid media is a solution containing nerve growth factors essential to a propagation of neuronal cells.

32. The apparatus, as recited in claim 24, wherein said neurite sustaining fluid media is a solution containing nerve growth factors essential to a propagation of neuronal cells.

33. A method for purification and assay of neurites, comprising said steps of:
(a) providing a pore-sized controlled porous filter membrane which outspace side surface is coated with a cell adhesion layer to form an adhesion surface; and
(b) combining said neuronal cells with said porous filter membrane in an aqueous environment under conditions in which outgrown neurites from cell bodies of said neuronal cells are attracted to and grow on said adhesion surface.

34. The method, as recited in claim 33, wherein said outgrown neurites emerging from said cell bodies of said neuronal cells pass through pores provided in said porous filter membrane while each of said pores has a size smaller than said cell bodies of said neuronal cells so as to prevent said cell bodies of said neuronal cells passing through said pores of said porous filter membrane, wherein said outgrown neurites traverse said pores to attach and track across said adhesion surface.

35. The method, as recited in claim 33, further comprising the steps of:
(c) culturing said neuronal cells on an opposing side surface of said adhesion surface of said porous filter membrane with a nerve growth factor (NGF) as a culture media; and
(d) separating said outgrown neurites from said cell bodies of said neuronal cells.

36. The method, as recited in claim 35, wherein said outgrown neurites is separated from said cell bodies by scraping said opposing side surface until said neurites are purifiable from said adhesion surface of said porous filter membrane.

37. The method, as recited in claim 35, after the step (d), further comprising a step of:
(e) analyzing said neurites on said porous filter membrane with immunohistochemical staining assays and/or in situ hybridization.

38. The method, as recited in claim 35, after the step (d), further comprising a step of:
(e) analyzing subcellular contents of said neurites by isolating a lysate of said purified neurites free of said cell bodies.

39. The method, as recited in claim 34, wherein said pore of said filter membrane has a size of 0.8 to 10 microns.

40. The method, as recited in claim 33, wherein said cell adhesion layer is a material coated on said outspace side surface of said porous filter membrane, wherein said material is selected from a group consisting a cell adhesion factor, a positively-charged molecule, an electrostatic charged material, and a combination of said cell adhesion factor, said positively-charged molecule and said electrostatic charged material.

41. The method, as recited in claim 40, wherein said cell adhesion factor is selected from a group consisting of fibronectin, laminin, collagen, vitronectin and tenascin, and active fragments and synthetic analogs having a cell binding domain thereof, and a combination thereof.

42. The method, as recited in claim 40, wherein said cell adhesion factor is a laminin.

43. The method, as recited in claim 40, wherein said positively-charged molecule is selected from a group consisting of polylysine, chitosan, poly(ethyleneimine), and acrylics polymerized from acrylamide or methacrylamide and incorporating positively-charged groups in the form of primary, secondary, or tertiary amines, or quaternary salts and a combination thereof.

44. The method, as recited in claim 40, wherein said electrostatic charged material is selected from a group consisting of poled polytetrafluoethylene (PFE), poled polyvinylidene fluoride (PVDF), fluoroethylenepolypropylene (FEP) and a combination thereof.

45. The method, as recited in claim 40, wherein said electrostatic charged material is created by electrostatic charges induced asymmetrically of the membrane composition by means of plasma or corona discharge.

46. The method, as recited in claim 33, wherein said porous filter membrane is selected from a group consisting of polycarbonate, polystyrene and polypropylene.

47. The method, as recited in claim 33, wherein said cell adhesion layer extends to a pore internal surface of each of said pores of said porous filter membrane
